(19) United States
(12) Patent Application Publication
(10) Pub. No.: US 2009/0173348 A1
(43) Pub. Date: Jul. 9, 2009

(54) Method And Apparatus For Inducing And Controlling Hypoxia

(76) Inventors: Joseph A. Fisher, Thornhill (CA); Alex Vesely, Toronto (CA); Hiroshi Sasano, Aichi (JP); Steve Iscoe, Kingston (CA); Ronald Somogyi, Toronto (CA); Eitan Prisman, Toronto (CA)

Correspondence Address: KLEIN, O'NEILL & SINGH, LLP, 43 CORPORATE PARK, SUITE 204, IRVINE, CA, 92606, US

(21) Appl. No.: 11/817,062

(22) PCT Filed: Feb. 24, 2006
(86) PCT No.: PCT/CA2006/000284

§ 371 Date: Mar. 3, 2009

Related U.S. Application Data
(60) Provisional application No. 60/656,584, filed on Feb. 25, 2005.

(51) Int. Cl. A61M 16/22

(52) U.S. Cl. 128/205.12

(57) ABSTRACT

An apparatus for inducing hypoxia in a subject is provided. The apparatus includes a breathing port, an inspiratory reservoir, means for introducing oxygen into the apparatus, means for controlling the flow rate of entry of oxygen into the apparatus at a rate below the subject's metabolic requirements, an expiratory reservoir having a vent, Sequential Gas Delivery means, and means for removing CO₂ from the circuit. The Sequential Gas Delivery means are for directing the gases such that upon expiration, the subject expires into the expiratory reservoir, and, upon inspiration, subject inspires first from the inspiratory reservoir, and, on any breath, once said inspiratory reservoir is depleted, gas for the balance of that inspiration is delivered from the expiratory reservoir

### FIELD OF THE INVENTION

This invention relates generally to a method and apparatus for inducing and controlling hypoxia.

### BACKGROUND OF THE INVENTION

There are numerous situations in which to induce hypoxia in a person. For example, in hypoxic training, air having a lower partial pressure of oxygen (PO₂) than ambient air is breathed for a period of time. Scientific studies have shown intermittent hypoxic training causes physiological changes that can benefit athletic performance. Hypoxic training is also used as a pre-conditioning technique prior to exposure to high altitude conditions in order to minimize the possibility of developing high altitude sickness, as well as for preconditioning of organs such as the heart, brain kidney or liver prior to hypoxic insults during surgery.

Several patents have described apparatuses to produce hypoxic gas which can be breathed by the user. U.S. Pat. No. 5,467,764 discloses a hypobaric sleeping chamber. U.S. Pat. No. 5,964,222 discloses a hypoxic tent and U.S. Pat. No. 5,799,652 discloses a Hypoxic Room System. In all of these systems, the subject is placed inside a chamber, which is neither convenient, nor comfortable. More complex methods employ computer controlled orifices that adjust, based on feedback from the user's physiological inputs, the rate of mixing of ambient air. All of these systems require complex equipment such as oxygen concentrators, sensing equipment, and control feedback systems. Some commercial products use rebreathed gas mixed with ambient air to provide a hypoxic mixture. However, in some systems, the harder the subject breathes, the less hypoxic the gas mixture. This is exacerbated by the fact that hypoxia induces hyperventilation in most subjects.

### SUMMARY OF THE INVENTION

In an embodiment, the invention disclosed herein comprises a simple apparatus and method for reliably inducing hypoxia, and maintaining hypoxia at a fixed level regardless of how hard the subject breathes. Furthermore, in some exemplary embodiments, no electronics or power is required, although they may be used optionally.

In an embodiment, the subject breathes through a sequential gas delivery (SGD) circuit. In such a circuit, gas enters the inspiratory side of the circuit and is generally collected in an inspiratory reservoir. The subject expires into an expiratory reservoir, which ultimately leads to a vent exiting the circuit. Upon inspiration, the subject inspires first from the inspiratory reservoir, and if this reservoir is depleted and the subject is still inspiring, the balance of inspiration is taken from the expiratory reservoir. For the purposes of the applicant's teachings, the terms “depleted” and “empty” refer to the situation where no further gas can be obtained from the inspiratory reservoir without significant exertion and significant reduction of pressure in the circuit. Thus, a vessel can be referred to as ‘depleted’ or ‘empty’ even though the vessel still may contain some quantity of gas.

In an embodiment, the SGD has a means for removing CO₂ in gas breathed by the subject, such as a CO₂ scrubbing canister known in the art. Flow of gas into the inspiratory reservoir may be driven passively, by the reservoir containing a self inflating mechanism capable of entraining ambient air. Alternately, fresh gas flow may be directed to the circuit via a pump or blower. A flow control on the entry port of the inspiratory mechanism controls the rate of fresh gas flow entering the circuit. By setting the flow at various levels below the subject's alveolar ventilation requirement, the oxygen concentration in the inspired air is controlled. Furthermore, because the gas is delivered sequentially (first from the inspiratory reservoir, then from the expiratory reservoir), all of this hypoxic mixture is delivered to the alveoli. Hyperventilation does not change the subject's O₂ level because any gas inspired above the rate of entrainment of ambient air comes from the expiratory reservoir, which has the same composition as alveolar gas after gas exchange has occurred in the lung.

Where fresh gas is provided into the apparatus, the fresh gas may be provided by:

a) providing ambient air (which has 21% O₂ concentration) to the circuit at a gas flow rate lower than the subject's alveolar ventilation,
b) providing a higher concentration of O₂ than ambient air in the gas flow entering the circuit at a lower flow rate than in a), and
c) providing a lower concentration of O₂ than ambient air in the gas flow entering the circuit at a higher flow rate than in a),

provided that in each case, less total oxygen is delivered to the circuit than the subject's metabolic requirements at the time. For the purposes herein, the terms fresh gas and fresh gas flow rate refer to any of the provisions of gas outlined in a), b), and c) above.

In another embodiment, the invention is directed to a method of inducing hypoxia in a subject comprised of:

Providing to the subject an apparatus in accordance with any of the apparatus embodiments described herein;

Estimating or measuring the subject's alveolar ventilation; and

Reducing the rate of entry of air into the apparatus below the subject's alveolar ventilation.

In another embodiment, the invention is directed to a method of inducing hypoxia in a subject comprised of:

Providing to the subject an apparatus in accordance with any of the apparatus embodiments described herein;

Estimating or measuring the subject's oxygen consumption; and

Reducing the rate of entry of air into the apparatus below the subject's oxygen consumption.

In another embodiment, the invention is directed to an apparatus for inducing hypoxia in a subject comprising a breathing port, at least one inspiratory reservoir, an oxygen source for introducing oxygen into the apparatus, a flow rate controller controlling the flow rate of entry of oxygen into the apparatus at a rate below the subject's metabolic requirements, at least one expiratory reservoir at least one of which has a vent, a Sequential Gas Delivery (SGD) device, and a CO₂ removal device for removing CO2 from the Sequential Gas Delivery (SGD) device. The Sequential Gas Delivery (SGD) device is for directing the gases such that upon expiration, the subject expires into the at least one expiratory reservoir, and, upon inspiration, subject inspires first from the at least one inspiratory reservoir, and, on any breath, once said at least one inspiratory reservoir is depleted, gas for the balance of that inspiration is delivered from the at least one expiratory reservoir.

### BRIEF DESCRIPTION OF THE DRAWINGS

FIG. 1 shows a sequential gas delivery circuit with crossover limb configuration and weighted bellows as inspiratory reservoir, in accordance with an embodiment of the present invention.

FIG. 2 shows a sequential gas delivery circuit with the CO₂ removal material on the expiratory limb, in accordance with another embodiment of the present invention.

FIG. 3 shows a sequential gas delivery circuit with separate inspiratory and expiratory paths, with the CO₂ removal material on the rebreathing limb, in accordance with another embodiment of the present invention.

FIG. 4 shows a sequential gas delivery circuit with a pump capable of introducing fresh gas into the circuit, in accordance with another embodiment of the present invention.

FIG. 5 shows an alternative connection between an oxygen inlet to the apparatus shown in FIG. 1, and a source of oxygen.

FIG. 6 shows a controller and oxygen saturation measurement device operatively connected to a variable resistance for controlling the flow of oxygen into the apparatus shown in FIG. 1.

FIG. 7 shows a sequential gas delivery circuit with CO₂ removal material on an inspiratory limb, in accordance with another embodiment of the present invention.

FIG. 8 shows a sequential gas delivery circuit apparatus with a plurality of inspiratory reservoirs and a plurality of expiratory reservoirs, in accordance with another embodiment of the present invention.

### DETAILED DESCRIPTION OF THE INVENTION

This invention will be further understood in view of the following detailed description of exemplary embodiments.

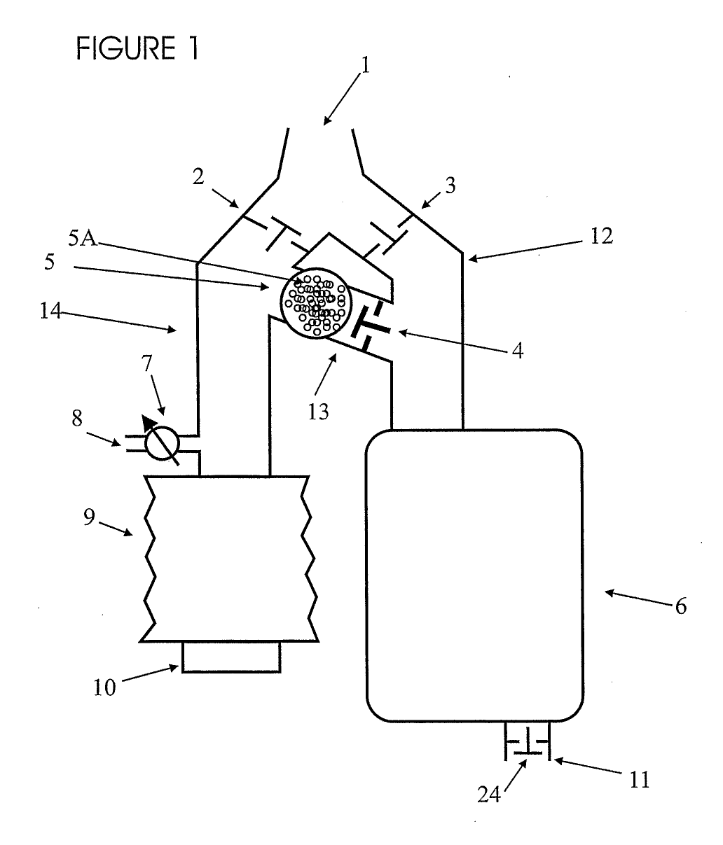

FIG. 1 shows an embodiment of the present invention. Subject breathes on the apparatus through subject port 1. The hypoxia breathing circuit is comprised of inspiratory limb 14 and expiratory limb 12, said limbs connected by crossover limb 13. Inspiratory limb 14 transports substantially all gas for breathing to the subject. Inspiratory limb 14 contains a one way valve 2 directed toward the subject. Expiratory limb 12 contains a one way valve 3 directed away from the subject towards expiratory reservoir 6. Crossover limb 13 contains a one way crossover valve 4 directed toward the inspiratory limb. One way valve 4 opens at a first differential pressure, which is greater than the second differential pressure required to open the one way valve 2 inspiratory. Port 8 is open to ambient air. Ambient air enters the circuit through port 8 at a flow rate determined by variable resistance 7 and the pressure generated in inspiratory reservoir 9 by the pull of mass 10 on the bottom of the reservoir 9. Reservoir 9 is preferably a bellows. Pressure generated by mass 10 is preferably less than opening pressure of crossover valve 4. A CO₂ removal device or means 5 removes CO₂ from rebreathed gas. Expired gas leaves the circuit via vent 11, which may optionally contain a one-way valve 24 directed toward the exit. Expiratory reservoir 6 preferably has high compliance and is large enough so that gas drawn from the expiratory side of the circuit comes from the reservoir 6 as it collapses and shrinks, and not from ambient air via vent 11. The expiratory reservoir 6 may be made, for example, from a suitably thin polymeric material.

The port 8 constitutes an oxygen inlet for the apparatus, or alternatively can be referred to as a means for introducing oxygen into the apparatus.

The optional variable resistance 7 may also be referred to as a flow rate controller 7 controls the rate of entry of oxygen into the apparatus, and can also be referred to as a means for controlling the flow rate of entry of oxygen into the apparatus. The flow rate controller 7 may be, for example, a Voltage Sensitive Orifice (VSO). Alternatively, any other suitable flow rate controller for controlling the rate of entry of oxygen into the apparatus or means for controlling the flow rate of entry of oxygen into the apparatus may be used.

The CO2 removal device or means 5 may be a commercially available CO2 scrubber known in the art. The CO2 removal device or means 5 may include a CO2 removal material 5A, such as soda lime, for absorbing CO2. Other materials 5A are also usable however, such as, for example, a zeolyte. Alternatively, any other suitable CO2 removal device or means 5 may be used.

The function of the circuit is as follows. The alveolar ventilation of the subject may be determined, for example, using the method disclosed by Preiss et. al. in U.S. patent application Ser. No. 10/135,655 published as US Patent Publication No. 2002-0185129 or is estimated from known values based on physiological parameters such as sex, weight, height, etc. Mass 10 causes constant negative pressure in inspiratory reservoir 9, drawing ambient air into port 8 at a rate controlled by resistance 7. Resistance 7 is set so that the flow is equal to the desired fraction of the subject's alveolar ventilation to achieve the desired hypoxic level. The subject inspires from inspiratory reservoir 9. When reservoir 9 is depleted, if the subject is still inspiring, pressure in the inspiratory limb 14 will become further reduced until valve 4 opens, allowing the subject to breath previously exhaled gas. To prevent CO₂ buildup, the CO2 scrubber 5 is positioned in the crossover limb and removes CO₂ from gas passing through crossover limb 13 for inspiration by the subject. Upon expiration, one way valve 3 opens allowing expired gas to enter the expiratory reservoir 6. If the expiratory reservoir is filled, further expiration vents via vent 11.

Instead of measuring or estimating the subject's alveolar ventilation, the method could include, for example, measuring or estimating the subject's oxygen consumption.

The Sequential Gas Delivery (SGD) circuit can also referred to as a Sequential Gas Delivery (SGD) device, or as a Sequential Gas Delivery (SGD) means. Alternatively, any other suitable Sequential Gas Delivery (SGD) device or means may be used.

It should be noted that numerous variations on the embodiment described above are possible. For example, inspiratory reservoir 9 and mass 10 could be replaced with a different passive method of entrainment. For example, mass 10 could be replaced by a constant spring mechanism that opens the reservoir with a constant force. Alternately, self-inflating foam inside the reservoir could be used. Any self inflating container capable of creating a constant negative pressure is suitable.

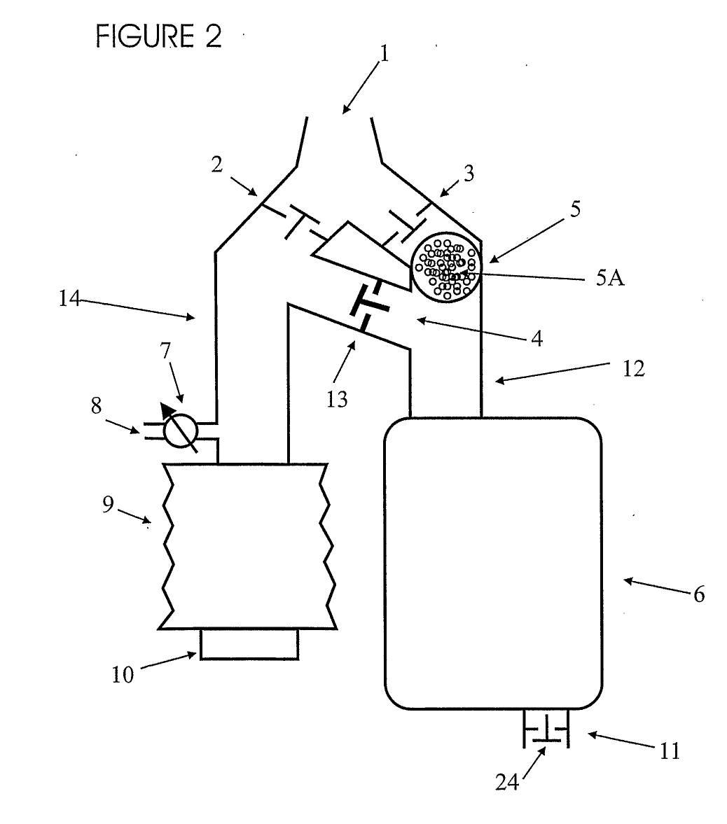

Another exemplary embodiment is shown in FIG. 2. In this circuit, scrubber 5 is positioned within the expiratory limb 12 and is positioned to receive substantially all of the expired gas before the gas enters the expiratory reservoir 6. Many types of flow resistances and flow controls to control the rate of entrainment of ambient air are known to those skilled in the art.

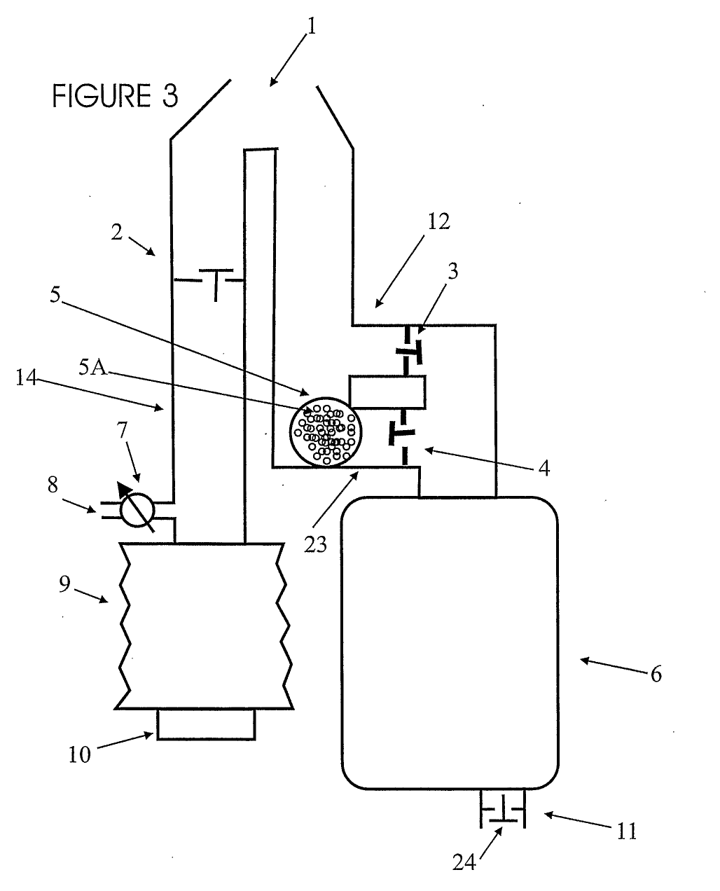

Many of the sequential gas delivery circuits described by Fisher et. al. in Canadian Patent application 2,419,575, which is incorporated herein by reference, are suitable for use with the present invention. For example SGD circuits described in FIGS. 3B, 3C, 3D, 3E, 5B, 5C, 5A, and 6A of the '575 application would be suitable, as long as a flow control means capable of setting the fresh gas flow rate into the inspiratory reservoir below the alveolar ventilation of the subject is provided.

As an example, FIG. 3 herein shows a further exemplary embodiment of a hypoxia apparatus using a sequential gas delivery circuit wherein instead of a crossover limb between inspiratory and expiratory limbs, there is a bypass limb 23 through which rebreathed gas is inspired. The CO₂ scrubber 5 would preferably be on this limb, although it could also be on expiratory limb 12.

In this embodiment, the one way bypass valve, shown at 4, opens at a first differential pressure, which is greater than the second differential pressure required to open the one way inspiratory valve 2.

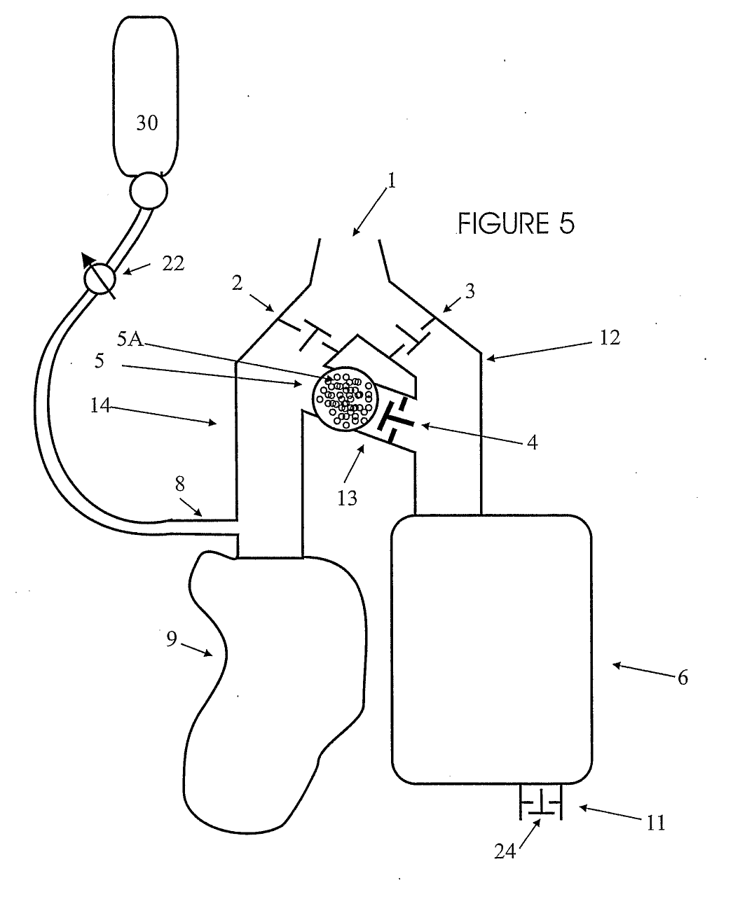

Referring to FIG. 5, the oxygen inlet 8 in any of the embodiments shown and described herein may be connected to a source of oxygen 24. The oxygen source 24 provides a gas with a concentration of oxygen that may be greater than or less than the concentration of oxygen in ambient air, or may alternatively provide a gas with oxygen in the same concentration as ambient air.

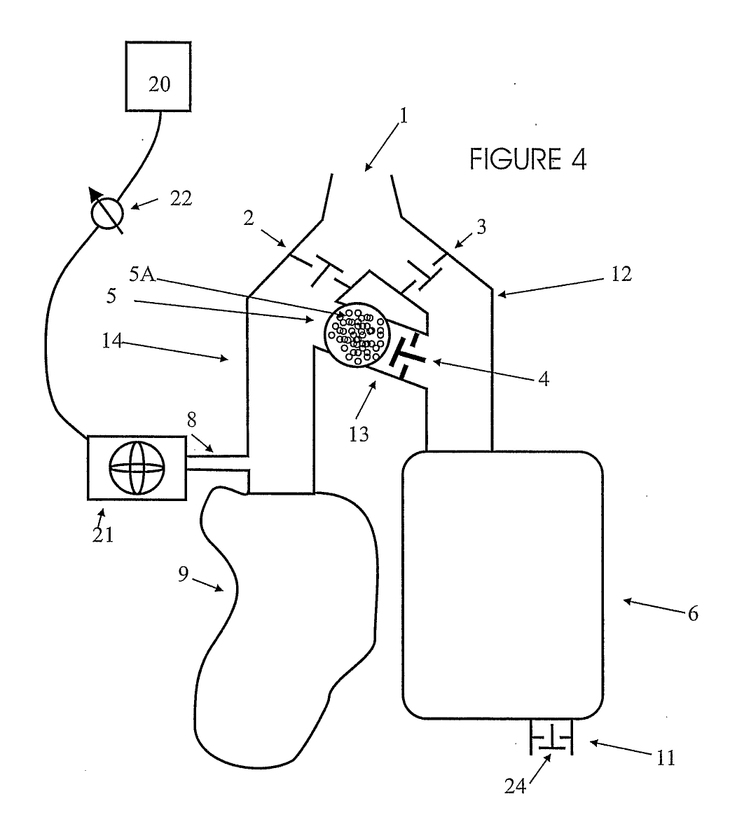

FIG. 4 shows a further exemplary embodiment of the present invention. In FIG. 4, a preferably adjustable pump 21 capable of pumping a desired rate of gas (eg. ambient air) is connected to fresh gas port 8, also referred to as the oxygen inlet 8. With such an embodiment, the inspiratory reservoir 9 may be a simple bag. Pump 21 speed may optionally be adjusted via controller 22 which may be further controlled by an optional oxygen saturation measurement device or means 20, which would preferably be a pulse oximeter but could be any other suitable oxygen saturation measurement device or means. When used in this configuration, the oxygen saturation measurement device or means 20 would measure the subject's oxygen saturation and send output relating to the measurements to the controller. The controller 22 would compare the saturation to the saturation required to achieve the desired hypoxic level. Controller 22 would adjust the speed of the pump 21 up or down to provide the required fresh gas flow based on the comparison. Thus, the pump 21 acts as a flow rate controller in embodiments wherein its speed is variable.

It will be appreciated that the rate of entry of oxygen into the apparatus shown in FIG. 4 is controlled by the pump 21 based on output from the oxygen saturation measurement device 20 and based on the target oxygen saturation selected for the subject.

The pump 21 may be used to provide air to an inspiratory reservoir 9 in any of the embodiments described herein, such as, for example, the embodiments shown in FIGS. 2 and 3. In any such embodiments, a self-inflating reservoir could be replaced by a reservoir similar to the reservoir 9 shown in FIG. 4. Optionally, the controller 22 could be used in these embodiments also. As a further option, the oxygen saturation measurement device or means 20 could be used in these embodiments also.

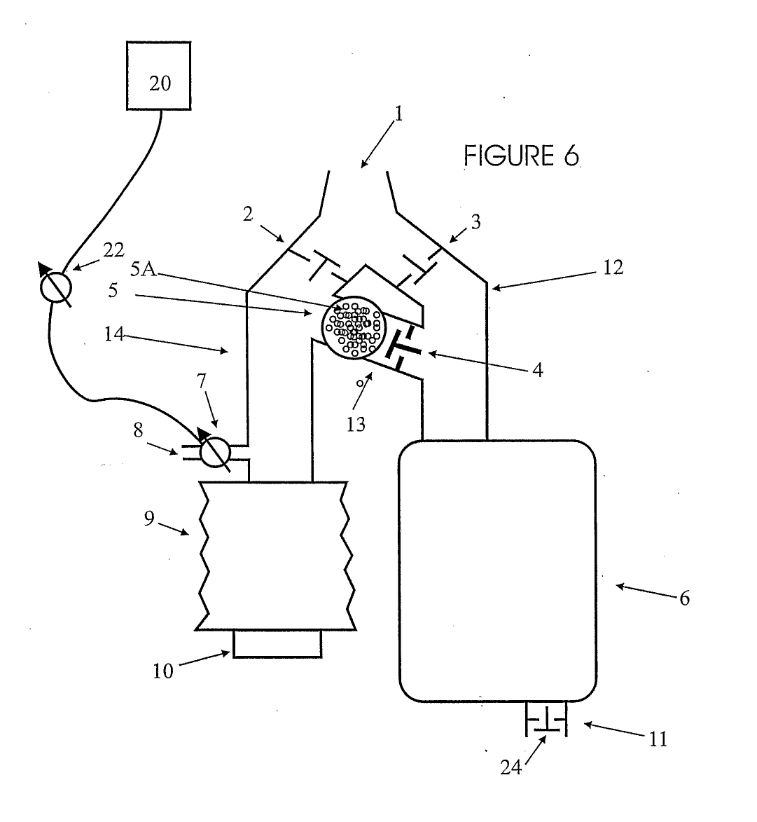

Reference is made to FIG. 6. As yet another alternative, the oxygen saturation measurement device or means 20 and controller 22 could be used with any of the embodiments shown herein without a pump. The controller 22 could control the variable resistance 7 to control the rate of entry of oxygen into the apparatus, based on the output from the oxygen saturation measurement device or means 20 to the controller 22 and based on the target oxygen saturation for the subject. The oxygen inlet 8 in this embodiment could be connection either to ambient air, or to a source of oxygen, such as a pressurized tank.

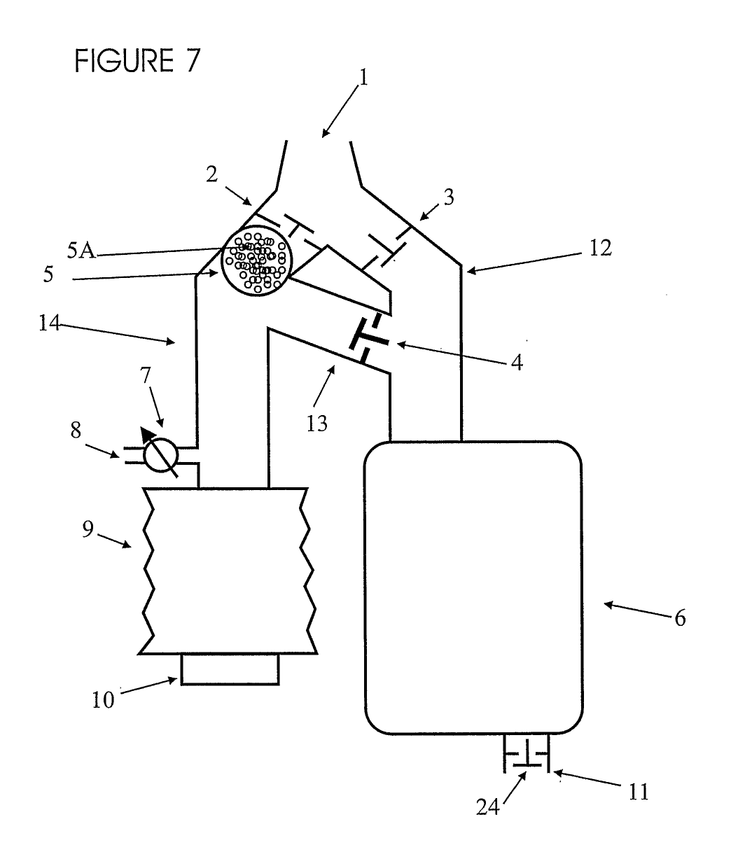

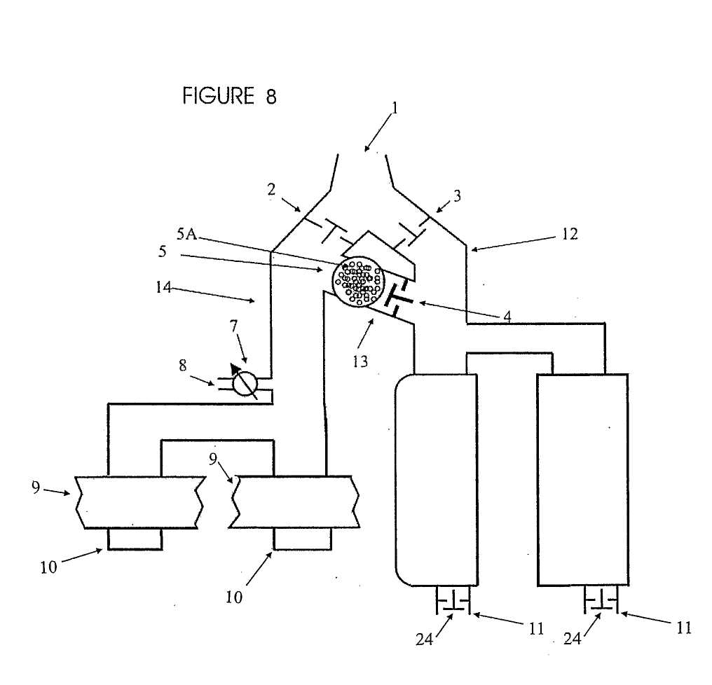

In the embodiment shown in FIG. 8, the reservoir 9 is preferably a bellows, however, other structures may be alternatively suitable. Reference is made to FIG. 7, which shows the apparatus with the CO2 removal device or means 5 on the inspiratory limb 14. In this embodiment, all of the gas inspired by the subject passes through the CO2 scrubber.

Reference is made to FIG. 8. It is optionally possible for the apparatus to include a plurality of inspiratory reservoirs 9 instead of just one, independent of the number of expiratory reservoirs 6 the apparatus has. Separately, it is optionally possible for the apparatus to include a plurality of expiratory reservoirs 6 instead of just one, independent of the number of inspiratory reservoirs 9 the apparatus has.

Provided the detailed disclosure herein, those skilled in the art may envision how the present invention could be practiced using alternative embodiments and variations thereof. The foregoing detailed description should be regarded as illustrative rather than limiting.

1. An apparatus for inducing hypoxia in a subject, comprising:
a) a breathing port;
b) an inspiratory reservoir;
c) an oxygen inlet for introducing oxygen into the apparatus;
d) a flow rate controller controlling the flow rate of entry of oxygen into the apparatus at a rate below the subject's metabolic requirements;
e) an expiratory reservoir having a vent;
f) a Sequential Gas Delivery device operable for directing respiratory gases such that upon expiration, the subject expires into the expiratory reservoir, and, upon inspiration, subject inspires first from the inspiratory reservoir, and, on any breath, once said inspiratory reservoir is depleted, gas for the balance of that inspiration is delivered from the expiratory reservoir; and
g) a CO₂ removal device operable for removing CO₂ from the Sequential Gas Delivery device.

2. The apparatus of claim 1, wherein, in use, the oxygen inlet is open to ambient air.

3. The apparatus of claim 1, wherein, in use, the oxygen inlet is connected to a source of gas that contains a different concentration of oxygen than ambient air.

4. The apparatus of claim 1, wherein the inspiratory reservoir is a bellows.

5. The apparatus of claim 1, wherein the oxygen inlet is open to a source of oxygen and wherein a reduction in pressure in the inspiratory reservoir generates a gas flow from the source of oxygen into the apparatus through the oxygen inlet.

6. The apparatus of claim 5, wherein the oxygen inlet is open to ambient air.

7. The apparatus of claim 1, wherein the inspiratory reservoir is self inflating.

8. The apparatus of claim 5, further comprising a mass having a selected weight connected to the inspiratory reservoir, wherein the weight of the mass urges the inspiratory reservoir to increase in volume.

9. The apparatus of claim 1, wherein the Sequential Gas Delivery Device has an exit, and the expiratory reservoir includes a one way valve in the vent directed toward the exit of the Sequential Gas Delivery device.

10. The apparatus of claim 1, wherein a pump is connected to the oxygen inlet for pumping oxygen to the oxygen inlet.

11. The apparatus of claim 1, wherein the Sequential Gas Delivery device comprises a one way valve directed toward the subject in an inspiratory limb connecting the inspiratory reservoir to the subject, a one way valve directed toward the expiratory reservoir in an expiratory limb connecting the expiratory reservoir to the subject, and a one way crossover valve in a crossover limb connecting the inspiratory and expiratory limbs, wherein the one way crossover valve is directed toward the inspiratory limb, wherein the one way crossover valve opens at a first differential pressure and wherein the one way inspiratory valve opens at a second differential pressure, and wherein the first differential pressure is greater than the second differential pressure.

12. The apparatus of claim 1 wherein the Sequential Gas Delivery device comprises a one way valve directed toward the subject in an inspiratory limb connecting the inspiratory reservoir to the subject, a one way valve directed toward the expiratory reservoir in an expiratory limb connecting the expiratory reservoir to the subject, and a one way bypass valve in a bypass limb connected to the expiratory limbs on both sides of the one way expiratory valve, directed toward the subject, wherein said one way bypass valve opens at a first differential pressure and wherein the one way inspiratory valve opens at a second differential pressure, and wherein the first differential pressure is greater than the second differential pressure.

13. The apparatus of claim 1, wherein the CO₂ removal device includes a CO2 absorbing material.

14. The apparatus of claim 1, wherein the CO₂ removal device is disposed to receive substantially all expired gas.

15. The apparatus of claim 1, wherein the CO₂ removal device is disposed to receive only gas passing from the expiratory reservoir to the subject.

16. The apparatus of claim 1, wherein the sequential gas delivery includes an inspiratory limb for transporting substantially all gas for breathing to the subject and wherein the CO₂ removal device is disposed on the inspiratory limb.

17. The apparatus of claim 1, further comprising an oxygen saturation measurement device for measuring the oxygen saturation of the subject, wherein the flow rate controller controls the rate of entry of oxygen into the apparatus based in part on output from the oxygen saturation measurement device.

18. The apparatus of claim 17, wherein the rate of entry of oxygen into the apparatus is controlled using output from the oxygen saturation measurement device to achieve a selected oxygen saturation in the subject.

19. The apparatus of claim 1, wherein the apparatus comprises a plurality of the inspiratory reservoirs.

20. The apparatus of claim 1, wherein the apparatus comprises a plurality of the expiratory reservoirs.

21. A method of inducing hypoxia in a subject, comprising;
a) Providing to the subject an apparatus as claimed in claim 1;
b) Estimating or measuring alveolar ventilation of the subject; and
c) Reducing the rate of entry of air into the apparatus below the alveolar ventilation of the subject based on the result of step b).

22. The method of claim 21, further comprising measuring the oxygen saturation of the subject, and setting the rate of entry of oxygen into the apparatus to achieve a selected oxygen saturation.

23. A method of inducing hypoxia in a subject, comprising:
a) Providing to the subject an apparatus as claimed in claim 1;
b) Estimating or measuring the oxygen consumption of the subject; and
c) Reducing the rate of entry of air into the apparatus below the oxygen consumption of the subject based on the result of step b).

24. A method of inducing hypoxia in a subject, comprising:
a. Providing a fresh gas flow containing a partial pressure of oxygen that is lower than the metabolic requirement of the subject,
b. Collecting expired gas from the subject;
c. Removing CO₂ from at least a portion of the expired gas to provide a substantially CO₂-free rebreathed gas stream;
d. Delivering a volume of the fresh gas to the subject; and
e. Delivering at least some of the substantially CO₂-free rebreathed gas stream to the subject after delivering the volume of fresh gas to the subject, based on the inspiratory need of the subject.
